(19) United States
(12) Patent Application Publication
(10) Pub. No.: US 2020/0261155 A1
(43) Pub. Date: Aug. 20, 2020

(54) IMAGE BASED ROBOT GUIDANCE

(71) Applicant: KONINKLIJKE PHILIPS N.V., EINDHOVEN (NL)

(72) Inventors: Aleksandra POPOVIC, BOSTON, MA (US); David Paul NOONAN, NEW YORK, NY (US)

(21) Appl. No.: 16/066,079

(22) PCT Filed: Dec. 21, 2016
(86) PCT No.: PCT/IB2016/057863

§ 371 Date: Jun. 26, 2018

Related U.S. Application Data
(60) Provisional application No. 62/272,737, filed on Dec. 30, 2015.

(51) Int. Cl. A61B 34/10; A61B 34/37; A61B 34/35; A61B 34/20

(52) U.S. Cl. CPC A61B 34/10; A61B 34/37; A61B 2034/2055; A61B 34/20; A61B 2034/107; A61B 34/35

(57) ABSTRACT

A method and system provide two light beams which intersect at a remote center of motion (RCM) of a robot having an end-effector at a distal end thereof; capture images of a planned entry point and a planned path through the RCM; register the captured images to three-dimensional pre-operative images; define an entry point and path for the RCM in the captured images using the light beams; detect and track in the captured images a reference object having a known shape; in response to information about the entry point, the path, and the reference object, compute robot joint motion parameters to align the end-effector to the planned entry point and planned path; and communicate the computed robot joint motion parameters to the robot to align the end-effector to the planned entry point and the planned path.

### TECHNICAL FIELD

This invention pertains a robot, a robot controller, and a method of robot guidance using captured images of the robot.

### BACKGROUND AND SUMMARY

Traditional tasks in surgery and interventions, such as laparoscopic surgery or needle placement for biopsy or therapy, include positioning of a rigid device (e.g. a laparoscope or a needle or other “tool”) through an entry point in the body along a path to a target location. To improve workflow and accuracy and allow consistent tool placement, these tasks may be performed by robots. These robots typically implement five or six degrees-of-freedom (e.g., three degrees of freedom for movement to the entry point, and two or three for the orientation of the tool along the path). Planning of the entry point and the path of the tool is typically done using 3D images that are acquired preoperatively, for example using computed tomography (CT), magnetic resonance imaging (MRI), etc.

In surgical operating rooms, 2D imaging modalities are typically available. They include intraoperative cameras, such as endoscopy cameras or navigation cameras, intraoperative 2D X-ray, ultrasound, etc. These 2D images can be registered to preoperative 3D images using a number of methods known in art, such as those disclosed in U.S. Patent Application Publication 2012/0294498 A1 or U.S. Patent Application Publication 2013/0165948 A1, which disclosures are incorporated herein by reference. Such registration allows a preoperative plan, which may include several incision points and tool paths, to be translated from preoperative to intraoperative images.

In existing systems and methods, a mathematical transformation between image coordinates and robot joint space has to be established to close the control loop between control of the robot and intraoperative images that hold information about the surgical plan.

The entire process is referred to as “system calibration” and requires various steps such as camera and robot calibration. Furthermore, to provide full calibration, depth between the camera and the organ/object under consideration needs to be measured either from images or using special sensors. Camera calibration is a process to establish inherent camera parameters: the optical center of the image, focal lengths in both directions and the pixel size. This is usually done preoperatively and involves acquisition of several images of a calibration object (usually a chessboard-like object) and computation of parameters from those images. Robot calibration is a process of establishing the mathematical relation between the joint space of the robot and the end-effector (an endoscope in this context).

However, the process to obtain system calibration involves several complications. For example, if some of the imaging parameters are changed during the surgery (e.g. camera focus is changed), the camera calibration needs to be repeated. Furthermore, robot calibration usually requires a technical expert to perform calibration. And if the user/surgeon moves an endoscope relative to the robot, calibration needs to be repeated. These complications are tied to many workflow pitfalls, including the need for technical training for operating room staff, prolonged operating room times, etc.

Accordingly, it would be desirable to provide a system and a method for image-based guidance of a multi-axis robot using intraoperative 2D images (e.g., obtained by endoscopy, X-ray, ultrasound, etc.) without a need for intraoperative calibration or registration of the robot to the imaging system.

In one aspect of the invention, a system includes: a robot having a remote center of motion (RCM) mechanism with two motor axes, and an end-effector at a distal end of the robot; a light projection apparatus configured to project light beams intersecting at the RCM; an imaging system configured to capture images of the RCM mechanism in a field of operation including a planned entry point and a planned path through the RCM; and a robot controller configured to control the robot and position the RCM mechanism, the robot controller including an image processor which is configured: to receive the captured images from the imaging system, to register the captured images to three-dimensional (3D) pre-operative images, to define an entry point and path for the RCM in the captured images using the projected light beams, and to detect and track in the captured images a reference object having a known shape, wherein the robot controller is configured to: compute robot joint motion parameters, in response to the defined entry point, the defined path, and the detected reference object, which align the end-effector to the planned entry point and the planned path; produce robot control commands, based on the computed robot joint motion parameters, which align the end-effector to the planned entry point and the planned path; and communicate the robot control commands to the robot.

In some embodiments, the image processor is configured to detect the entry point as an intersection of the projected light beams, and the robot controller is configured to control the robot to align the intersection of the projected light beams with the planned entry point.

In some embodiments, the image processor is configured to: project the known shape of the reference object at the planned entry point onto the captured images, segment the detected reference object in the captured images, and align geometric parameters of the segmented reference object in the captured images to geometric parameters of the projected known shape of the reference object at the planned entry point, and the robot controller is configured to control the robot to overlay the detected reference object in the captured images with the projected known shape.

In some embodiments, the imaging system is configured to capture two-dimensional (2D) images of the RCM mechanism in the field of operation from a plurality of cameras spaced apart in a known configuration, and the image processor is configured to detect and track the reference object having a known shape in the captured 2D images from each of the plurality of cameras, and to reconstruct a 3D shape for the reference object from the captured 2D images.

In some embodiments, the RCM mechanism is configured to rotate the end-effector about an insertion axis passing through the planned entry point, and the end-effector has a feature that defines its orientation in a plane perpendicular to the insertion axis, wherein the image processor is configured to detect the feature in the captured images and to project a planned position of the feature onto the captured images, and wherein the robot controller is configured to control the robot to align the detected feature and the planned position.

In some embodiments, the reference object is the end-effector.

In some versions of these embodiments, the imaging system includes a camera and an actuator for moving the camera, the camera is positioned by the actuator along the planned path, and the robot controller is configured to control a position of the end-effector so that the image processor detects a parallel projection of the end-effector.

In some embodiments, the imaging system includes an X-ray system configured to generate a rotational three-dimensional (3D) scan of the planned path.

In another aspect of the invention, a method comprises: providing at least two light beams which intersect at a remote center of motion (RCM) defined by an RCM mechanism of a robot having an end-effector at a distal end thereof; capturing images of the RCM mechanism in a field of operation including a planned entry point and a planned path through the RCM; registering the captured images to three-dimensional (3D) pre-operative images; defining an entry point and path for the RCM in the captured images using the projected light beams; detecting and tracking in the captured images a reference object having a known shape; in response to information about the entry point, the path, and the reference object, computing robot joint motion parameters which align the end-effector to the planned entry point and the planned path; and communicating robot control commands to the robot, based on the computed robot joint motion parameters, which align the end-effector to the planned entry point and the planned path.

In some embodiments, the method includes detecting the entry point as an intersection of the projected light beams, and controlling the robot to align the intersection of the projected light beams with the planned entry point.

In some embodiments, the method includes: projecting the known shape of the reference object at the planned entry point onto the captured images; segmenting the detected reference object in the captured images; aligning geometric parameters of the segmented reference object in the captured images to geometric parameters of the projected known shape of the reference object at the planned entry point; and controlling the robot to overlay the detected reference object in the captured images with the projected known shape.

In some embodiments, the method includes: capturing two-dimensional (2D) images of the RCM mechanism in the field of operation from a plurality of cameras spaced apart in a known configuration; and detecting and tracking the reference object having a known shape in the captured 2D images from each of the plurality of cameras; and reconstructing a 3D shape for the reference object from the captured 2D images.

In some embodiments, the method includes: rotating the end-effector about an insertion axis passing through the planned entry point, wherein the end-effector has a feature that defines its orientation in a plane perpendicular to the insertion axis; detecting the feature in the captured images; projecting a planned position of the feature onto the captured images; and controlling the robot to align the detected feature and the planned position.

In some embodiments, the method includes: capturing the images of the RCM mechanism using a camera positioned along the planned path, wherein the reference object is the end-effector; and controlling a position of the end-effector so that a parallel position of the end-effector is detected in the captured images.

In yet another aspect of the invention, a robot controller is provided for controlling a robot having a remote center of motion (RCM) mechanism with two motor axes and an end-effector at a distal end of the robot. The robot controller comprises: an image processor which is configured: to receive captured images of the RCM mechanism in a field of operation including a planned entry point and a planned path through the RCM, to register the captured images to three-dimensional (3D) pre-operative images, to define an entry point and path for the RCM in the captured images, and to detect and track in the captured images a reference object having a known shape; and a robot control command interface configured to communicate robot control commands to the robot, wherein the robot controller is configured to compute robot joint motion parameters, in response to the defined entry point, the defined path, and the detected reference object, which align the end-effector to the planned entry point and the planned path, and is further configured to produce the robot control commands, based on the computed robot joint motion parameters, which align the end-effector to the planned entry point and the planned path.

In some embodiments, the image processor is configured to detect the entry point as an intersection of the projected light beams, and the robot controller is configured to control the robot to align the intersection of the projected light beams with the planned entry point.

In some embodiments, the image processor is configured to: project the known shape of the reference object at the planned entry point onto the captured images, segment the detected reference object in the captured images, and align geometric parameters of the segmented reference object in the captured images to geometric parameters of the projected known shape of the reference object at the planned entry point, and the robot controller is configured to control the robot to overlay the detected reference object in the captured images with the projected known shape.

In some embodiments, the image processor is configured to receive two-dimensional (2D) images of the RCM mechanism in the field of operation from a plurality of cameras spaced apart in a known configuration, to detect and track the reference object having a known shape in the captured 2D images from each of the plurality of cameras, and to reconstruct a 3D shape for the reference object from the captured 2D images

In some embodiments, the RCM mechanism is configured to rotate the end-effector about an insertion axis passing through the planned entry point, the end-effector has a feature that defines its orientation in a plane perpendicular to the insertion axis, the image processor is configured to detect the feature in the captured images and to project a planned position of the feature onto the captured images, and the robot controller is configured to control the robot to align the detected feature and the planned position.

In some embodiments, the robot controller is configured to receive the captured images from a camera positioned by an actuator along the planned path, and the robot controller is configured to control a position of the end-effector so that the image processor detects a parallel projection of the end-effector.

### BRIEF DESCRIPTION OF THE DRAWINGS

FIG. 1 is a block diagram of one example embodiment of a robotic system.

FIG. 2 illustrates an exemplary embodiment of a robot control loop.

FIG. 3 illustrates one version of the embodiment of a robotic system of FIG. 1.

FIG. 4 is a flowchart illustrating major operations of one embodiment of a method of robot-based guidance.

FIG. 5 is a flowchart illustrating detailed steps of an example embodiment of a method of performing one of the operations of the method of FIG. 4.

FIG. 6 is a flowchart illustrating detailed steps of an example embodiment of a method of performing another one of the operations of the method of FIG. 4.

FIG. 7 illustrates an example of a captured video frame and an example overlay of a tool holder in the captured video frame.

FIG. 8 illustrates one example embodiment of a feedback loop which may be employed in an operation or method or robot-based guidance.

FIG. 9 illustrates a second version of the embodiment of a robotic system of FIG. 1.

FIG. 10 illustrates a third version of the embodiment of a robotic system of FIG. 1.

FIG. 11 illustrates a process of alignment and orientation of a circular robot tool holder to a planned position for the robot tool holder using a series of captured video frames.

FIG. 12 illustrates one example embodiment of another feedback loop which may be employed in an operation of method of robot-based guidance.

FIG. 13 illustrates a fourth version of the embodiment of a robotic system of FIG. 1.

### DETAILED DESCRIPTION

The present invention will now be described more fully hereinafter with reference to the accompanying drawings, in which preferred embodiments of the invention are shown. This invention may, however, be embodied in different forms and should not be construed as limited to the embodiments set forth herein. Rather, these embodiments are provided as teaching examples of the invention.

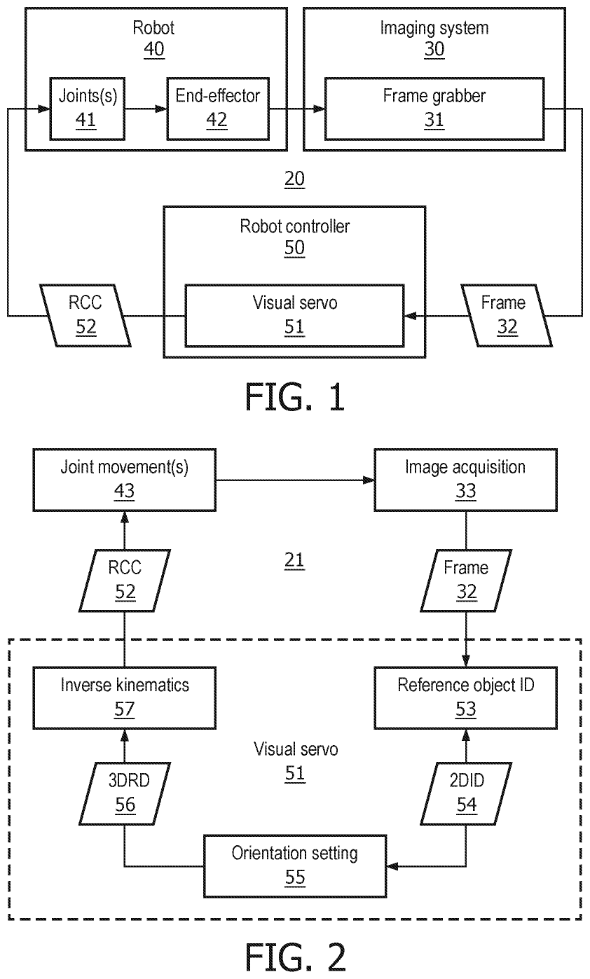

FIG. 1 is a block diagram of one example embodiment of a robotic system 20.

As shown in FIG. 1, a robotic system 20 employs an imaging system 30, a robot 40, and a robot controller 50. In general, robotic system 20 is configured for any robotic procedure involving automatic motion capability of robot 40. Examples of such robotic procedures include, but are not limited to, medical procedures, assembly line procedures and procedures involving mobile robots. In particular, robotic system 20 may be utilized for medical procedures including, but are not limited to, minimally invasive cardiac surgery (e.g., coronary artery bypass grafting or mitral valve replacement), minimally invasive abdominal surgery (laparoscopy) (e.g., prostatectomy or cholecystectomy), and natural orifice translumenal endoscopic surgery.

Robot 40 is broadly defined herein as any robotic device structurally configured with motorized control of one or more joints 41 for maneuvering an end-effector 42 of robot 40 as desired for the particular robotic procedure. End-effector 42 may comprise a gripper or a tool holder. End-effector 42 may comprise a tool such as a laparoscopic instrument, laparoscope, a tool for screw placement in spinal fusion surgery, a needle for biopsy or therapy, or any other surgical or interventional tool.

In practice, robot 40 may have a minimum of three (3) degrees-of-freedom, and beneficially five (5) or six (6) degrees-of-freedom. Robot 40 has a remote center of motion (RCM) mechanism with two motor axes intersecting the end-effector axis. Beneficially, robot 40 may have associated therewith a light projection apparatus (e.g., a pair of lasers) configured to project light beams (e.g., laser beams) along any of the axes of the RCM mechanism.

A pose of end-effector 42 is a position and an orientation of end-effector 42 within a coordinate system of robot 40.

Imaging system 30 may include one or more cameras. In some embodiments, imaging system 300 may include an intraoperative X-ray system which is configured to generate a rotational 3D scan. Imaging system configured to capture images of the RCM mechanism of robot 40 in a field of operation including a planned entry point for end-effector 42 or a tool held by end-effector 42 (e.g., for a surgical or interventional procedure), and a planned path for end-effector 42 or a tool held by end-effector 42 through the RCM.

Imaging system 30 may also include or be associated with a frame grabber 31. Robot 40 includes joints 41 (e.g., five or six joints 41) and an end-effector 42. As will be described in greater detail below, in some embodiments end-effector 42 is configured to be a tool holder to be manipulated by robot 40. Robot controller 50 includes a visual servo 51, which will be described in greater detail below.

Imaging system 30 may be any type of camera having a forward optical view or an oblique optical view, and may employ a frame grabber 31 of any type that is capable of acquiring a sequence of two-dimensional digital video frames 32 at a predefined frame rate (e.g., 30 frames per second) and capable of providing each digital video frame 32 to robot controller 50. Some embodiments may omit frame grabber 31, in which case imaging system 30 may just send its images to robot controller 50. In particular, imaging system 30 is positioned and oriented such that within its field of view it can capture images of end-effector 42 and a remote center of motion (RCM) 342 of robot 40, and an operating space in which RCM 342 is positioned and maneuvered. Beneficially, imaging system 30 is also positioned to capture images of a reference object having a known shape which can be used to identify a pose of end-effector 42. In some embodiments, imaging system 30 includes a camera which is actuated by a motor and it can be positioned along a planned instrument path for robot 40 once imaging system 30 is registered to preoperative images, as will be described in greater detail below.

Robot controller 50 is broadly defined herein as any controller which is structurally configured to provide one or more robot control commands (“RCC”) 52 to robot 40 for controlling a pose of end-effector 42 as desired for a particular robotic procedure by commanding definitive movements of each robotic joint(s) 41 as needed to achieve the desired pose of end-effector 42.

For example, robot control command(s) 52 may move one or more robotic joint(s) 41 as needed for facilitating a tracking of the reference object (e.g., end-effector 42) by imaging system 30 for controlling a set of one or more robotic joints 41 for aligning the RCM of robot 40 to a planned entry point for surgery, and for controlling an additional pair of robotic joints for aligning end-effector 42 with a planned path for surgery.

For robotic tracking of a feature of an image within digital video frames 32 and for aligning and orienting robot 40 with a planned entry point and planned path for end-effector 42 or a tool held by end-effector 42, robot controller 50 includes a visual servo 51 for controlling the pose of end-effector 42 relative to an image of the reference object identified in each digital video frame 32 and a projection of the reference object onto the image based upon its known shape and its position when the RCM is aligned and oriented with the planned entry point and path.

Toward this end, as shown in FIG. 2, visual servo 51 implements a reference object identification process 53, an orientation setting process 55 and an inverse kinematics process 57, in a closed robot control loop 21 with an image acquisition 33 implemented by frame grabber 31 and controlled movement(s) 43 of robotic joint(s) 41. In practice, processes 53, 55 and 57 may be implemented by modules of visual servo 51 that are embodied by any combination of hardware, software and/or firmware installed on any platform (e.g., a general computer, application specific integrated circuit (ASIC), etc.). Furthermore, processes 53 and 55 may be performed by an image processor of robot controller 50.

Referring to FIG. 2, reference object identification process 53 involves an individual processing of each digital video frame 32 to identify a particular reference object within digital video frames 32 using feature recognition algorithms as known in the art.

Referring again to FIG. 2, reference object identification process 53 generates two-dimensional image data (“2DID”) 54 indicating a reference object within each digital video frame 32, and orientation setting process 55 in turn processes 2D data 54 to identify an orientation or shape of the reference object. For each digital video frame 32 where the reference object is recognized, orientation setting process 55 generates three-dimensional robot data (“3DRD”) 56 indicating the desired pose of end-effector 42 of robot 40 relative to the reference object within digital video frame 32. Inverse kinematics process 57 processes 3D data 56 as known in the art for generating one or more robot control command(s) 52 as needed for the appropriate joint movement(s) 43 of robotic joint(s) 41 to thereby achieve the desired pose of end-effector 42 relative to the reference object within digital video frame 32.

In operation, the image processor of robot controller 50 may: receive the captured images from imaging system 30, register the captured images to three-dimensional (3D) pre-operative images, define an entry point and path for the RCM in the captured images using the projected light beams (e.g., laser beams), and detect and track the reference object in the captured images. Furthermore, robot controller 50 may: compute robot joint motion parameters in response to the defined entry point, the defined path, and the detected reference object, which align end-effector 42 to the planned entry point and the planned path; produce robot control commands 52 in response to the computed robot joint motion parameters, which align end-effector 42 to the planned entry point and the planned path; and communicate the robot control commands to robot 40.

Further aspects of various versions of robotic system 20 will now be described in greater detail.

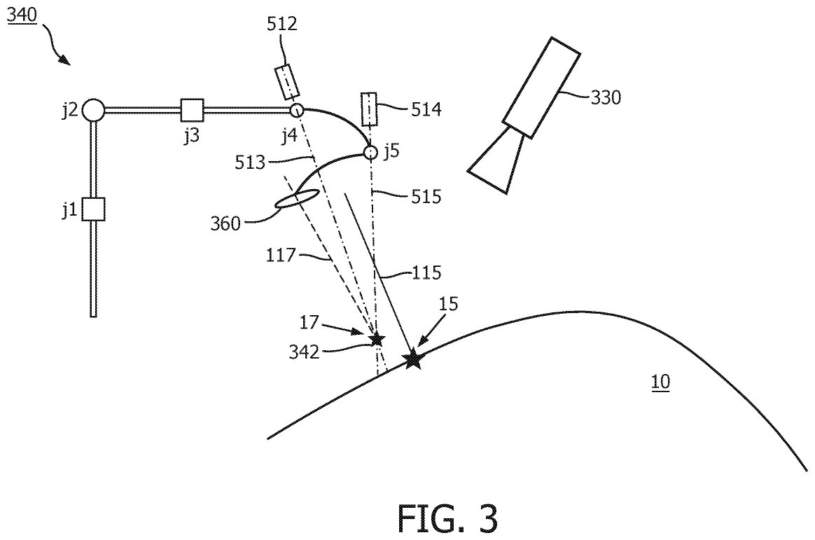

FIG. 3 illustrates a portion of a first version of robotic system 20 of FIG. 1. FIG. 3 shows an imaging device, in particular a camera, 330, and a robot 340. Here, camera 330 may be one version of imaging system 30, and robot 340 may be one version of robot 40. Camera 330 is positioned and oriented so that within its field of view it may capture images of at least portions of robot 340, including end-effector 42, and a remote center of motion (RCM) 342, and an operating space in which RCM 342 is positioned and maneuvered. Although not illustrated in FIG. 3, it should be understood that the robotic system illustrated in FIG. 3 includes a robot controller, such as robot controller 50 described above with respect to FIGS. 1 and 2.

Robot 340 has five joints: j1, j2, j3, j4 and j5, and an end-effector 360. Each of the joints j1, j2, j3, j4 and j5 may have an associated motor which can maneuver the joint in response to one or more robot control commands 52 received by robot 340 from a robot controller (e.g., robot controller 50). Joints j4 and j5 define RCM 342. First and second lasers 512 and 514 project corresponding RCM laser beams 513 and 515 in such a way that they intersect at RCM 342. In some embodiments, first and second lasers 512 and 514 project RCM laser beams 513 and 515 along the motor axes of joints j4 and j5. In an embodiment with a concentric arc system as illustrated in FIG. 3, first and second lasers 512 and 514 may be located anywhere along the arcs. Also shown are: a planned entry point 15 for subject 10 along a planned path 115, and a detected entry point 17 along a detected path 117.

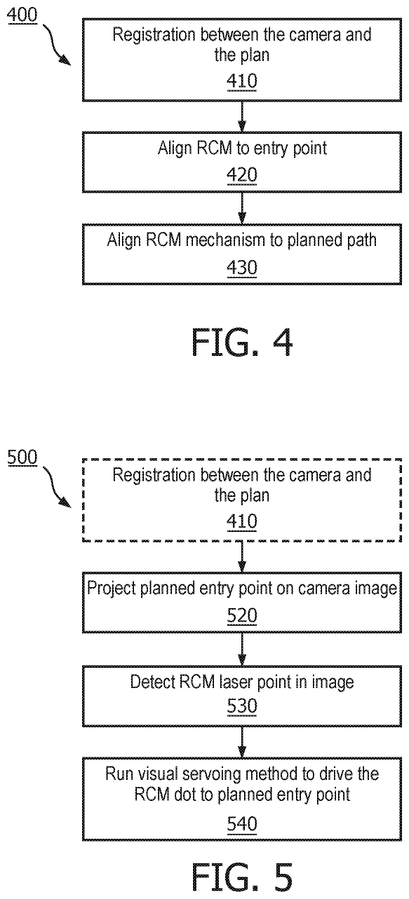

FIG. 4 is a flowchart illustrating major operations of one embodiment of a method 400 of robot-based guidance which may be performed by a robotic system. In the description below, to provide a concrete example it will be assumed that method 400 is performed by the version of robotic system 20 which is illustrated in FIG. 3.

An operation 410 includes registration of a plan (e.g., a surgical plan) for robot 340 and the camera 30. Here, the plan for robot 340 is described with respect to one or more preoperative 3D images. Accordingly, in operation 410 images (e.g., 2D images) produced by camera 300 may be registered to the preoperative 3D images using a number of methods known in art, including for example, methods described in Philips patent applications (e.g. US 2012/0294498 A1 or EP 2615993 B1).

An operation 420 includes aligning RCM 342 of robot 340 to planned entry point 15. Further details of an example embodiment of operation 420 will be described with respect to FIG. 5 below.

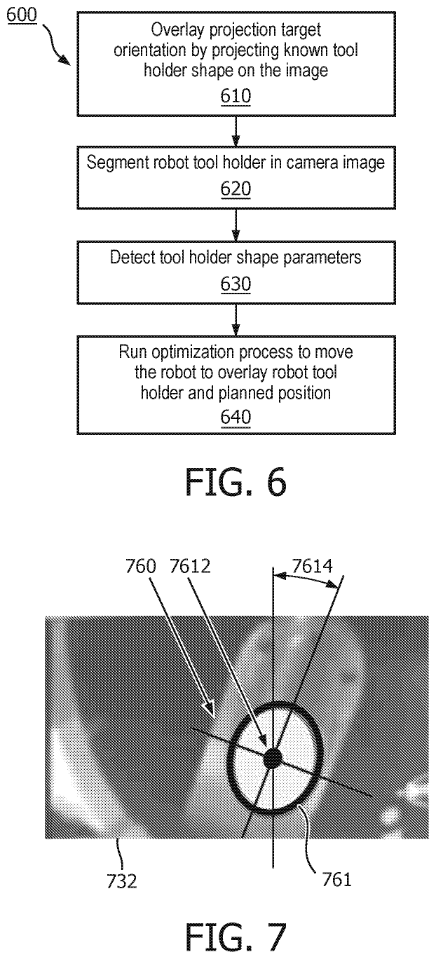

An operation 430 includes aligning the RCM mechanism (e.g., joints j4 and j5) of robot 340 to the planned path 117. Further details of an example embodiment of operation 430 will be described with respect to FIG. 6 below.

FIG. 5 is a flowchart illustrating detailed steps of an example embodiment of a method 500 for performing operation 420 of method 400. Here, it is assumed that an operation 410 for registration between preoperative 3D images and camera 300 has already been established.

In a step 520, an image processor or robot controller 50 projects a 2D point representing a 3D planned entry point 15 onto captured images (e.g., digital video frames 32) of camera 330. Since camera 330 is not moving with respect to subject 10, projected planned entry point 15 is static.

In a step 530, the intersection of RCM laser beams 513 and 515 can be detected in the captured images of camera 330 to define detected entry point 17. Beneficially, the robotic system and the method 500 make use of the fact that planned entry point 15 into subject 10 is usually on the surface of subject 10, and thus can be visualized by the view of camera 330 and projected onto the captured images, while the laser dots can be projected from lasers 512 and 514 are also be visible on subject 10 in the captured images to define detected entry point 17 for the current position and orientation of RCM 342 of robot 340.

In a step 540, robot controller 50 sends robot control commands 52 to robot 340 to move RCM 342 so as to drive entry point 17, defined by the intersection of RCM laser beams 513 and 515, to planned entry point 15. In some embodiments, step 540 may be performed by an algorithm described in U.S. Pat. No. 8,934,003 B2. Beneficially, step 540 may be performed with robot control commands 52 which direct movement of joints j1, j2 and j3. Beneficially, after defined entry point 17 is aligned with planned entry point 15, joints j1, j2, and j3 may be locked for subsequent operations, including operation 430. FIG. 6 is a flowchart illustrating detailed steps of an example embodiment of a method 600 for performing operation 430 of method 400. Here, it is assumed that an operation for registration between preoperative 3D images and camera 300 has already been established, as described above with respect to methods 400 and 500

In a step 610, an image processing subsystem of robot controller 50 overlays or projects onto the captured images (e.g., digital video frames 32) of camera 33 a known shape of a reference object as it should be viewed by camera when end-effector 42 is aligned to planned instrument path 115 and planned entry point 15. In the discussion to follow, to provide a concrete example it is assumed that the reference object is end-effector 42. However in general the reference object may be any object or feature in the field of view of camera 330 having a known size and shape. Here, image processing system is assumed to have a priori knowledge of the shape and size of end-effector 42. For example, if end-effector 42 has a circular shape, then its shape may be viewed in two dimensions by camera 330 as an ellipse, depending on the positional/angular relations between camera 330, end-effector 42, and planned entry point 15. In that case, the image processor may project or overlay onto captured images from camera 330 a target elliptical image representing the target position and orientation of end-effector 42 when end-effector 42 is aligned and oriented to planned entry point 15 along planned path 115. Furthermore, image processor 330 may define other parameters of the target elliptical image of end-effector 42 which may depend on the shape of end-effector 42, for example a center and an angle for the projected ellipse in the example case of a circular end-effector 42

In a step 620, the image processor detects and segments the image of end-effector 42 in the captured images.

In a step 630, the image processor detects a shape of the image of end-effector 42 in the captured images. Beneficially, image processor detects other parameters of the detected image end-effector 42 in the captured images, which may depend on the shape of end-effector 42. For example, assuming that end-effector 42 has a circular shape, yielding an elliptical image in the captured images of camera 330, then in step 630 the image processor may detect a center and an angle of the detected image of end-effector 42 in captured images 32.

FIG. 7 illustrates an example of a captured image 732 and an example projected overlay 760 of end-effector 42 onto captured image 732. Here it is assumed that projected overlay 760 represented the size and shape that end-effector 42 should have in a captured image of camera 330 when end-effector 42 is aligned and oriented to planned entry point 15 along planned path 115. In the example shown in FIG. 7, the center 7612 of projected overlay 760 of end-effector 42 is aligned with the center of the detected image of end-effector 42, but there exists a rotational angle 7614 between projected overlay 760 of end-effector 42 and the detected image of end-effector 42.

In that case, in step 640 robot controller 50 may execute an optimization algorithm to move robot 40, and in particular an RCM mechanism comprising joints j4 and j5, so as to align the image of end-effector 42 captured by camera with projected overlay 260. When the captured image of end-effector 42 is aligned with projected overlay 260, then end-effector 42 is aligned and oriented to planned entry point 15 along planned path 115.

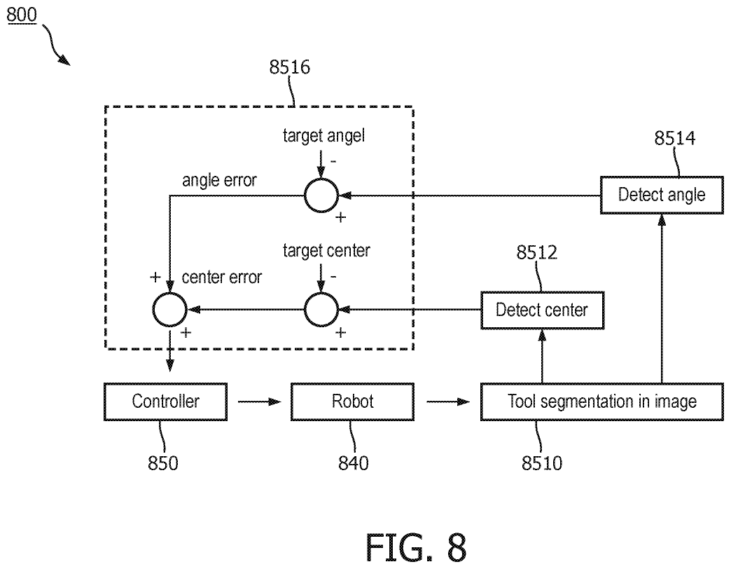

FIG. 8 illustrates one example embodiment of a feedback loop 800 which may be employed in an operation or method of robot-based guidance which may be executed, for example, by robotic system 20. Various operators of feedback loop 800 are illustrated as functional blocks in FIG. 8. Feedback loop 800 involves a controller 840, a robot 850, a tool segmentation operation 8510, a center detection operation 8512, an angle detection operation 8514, and a processing operation 8516. Here, feedback loop 800 is configured to operate with a reference object (e.g., end-effector 42) having an elliptical projection (e.g., a circular shape). In some cases, tool segmentation operation 8510, center detection operation 8512, angle detection operation 8514, and processing operation 8516 may be performed in hardware, software, firmware, or any combination thereof by a robot controller such as robot controller 50.

An example operation of feedback loop 800 will now be described.

Processing operation 8516 subtracts the detected center and angle of a captured image of end-effector 42 from a target angle and a target center for end-effector 42, resulting in two error signals: a center error and an angle error. Processing operation 8516 combines those two errors (e.g. adds them with corresponding weights) and supplies the weighted combination as a feedback signal to controller 850, which may be included as a component of robot controller 50 discussed above. Here controller 850 may be a proportional-integral-derivative (PID) controller or any other appropriate controller known in art, including a non-linear controller such as a model predictive controller. The output of controller 850 is a set of RCM mechanism joint velocities. The mapping to joint velocities can be done by mapping yaw and pitch of the end-effector 42 of robot 840 to x and y coordinates in the captured images. The orientation of end-effector 42 can be mapped using a homography transformation between the detected shape of end-effector 42 in the captured images, and the parallel projection of the shape onto the captured images.

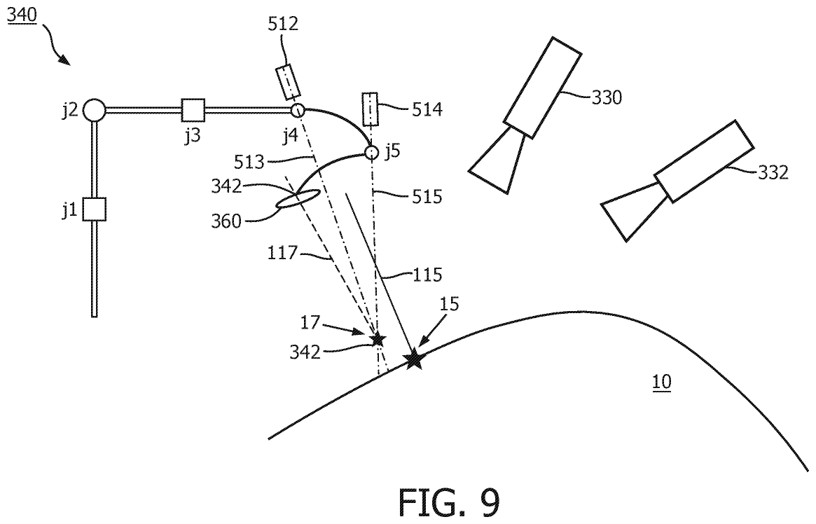

FIG. 9 illustrates a portion of a second version of robotic system 20 of FIG. 1. The second version of robotic system 20 as illustrated in FIG. 9 is similar in construction and operation to the first version illustrated in FIG. 3 and described in detail above, so for the sake of brevity only differences therebetween will now be described.

In the second version of robotic system 20, the image capturing system includes at least two cameras 330 and 332 spaced apart in a known or defined configuration. Each of the cameras 330 and 332 is positioned and oriented so that within its field of view it may capture images of at least portions of robot 340, including end-effector 42, and RCM 342, and an operating space in which RCM 342 is positioned and maneuvered. Accordingly, in this version of robotic system 20, the image processor may be configured to detect and track the reference object (e.g., end-effector 42) in the captured 2D images from each camera 330 and 332, and to reconstruct a 3D shape for end-effector 42 from the captured 2D images.

Here, the scale of the captured images can be reconstructed using a known size of end-effector 42 and focal lengths of cameras 330 and 332. Reconstructed position and scale will give a 3D position of robot 340 the coordinate frame of cameras 330 and 332. The orientation end-effector 42 can be detected using a homography transformation between the detected shape of end-effector 42 in the captured images, and the parallel projection of the shape onto the captured image. This version may reconstruct the position of robot 340 in 3D space and register the robot configuration space to the camera coordinate system. Robot control can be position based: the robot motors are moved in robot joint space to move end-effector 42 from an initial position and orientation to the planned position and orientation.

In another version of robotic system 20, the RCM mechanism is equipped with an additional degree of freedom such that is capable of rotating end-effector 42 around a tool insertion axis passing through planned entry point 15. Here also end-effector 42 is provided with a feature that defines its orientation in a plane perpendicular to the insertion axis, and the image processor is configured to detect the feature in the captured images and to project a planned position of the feature onto the captured images. For example, the feature could a circle or a rectangle with a pin. Robot controller 50 is configured to control robot 350 to align the detected feature and the planned position of the feature.

This version can be useful when end-effector 42 is not rotationally symmetric, e.g. end-effector 42 is a grasper or beveled needle. After both planned entry point 15 and orientation of end-effector 42 along path 115 are set, end-effector 42 is rotated using the additional degree of freedom until the planned and detected positions of the feature are aligned.

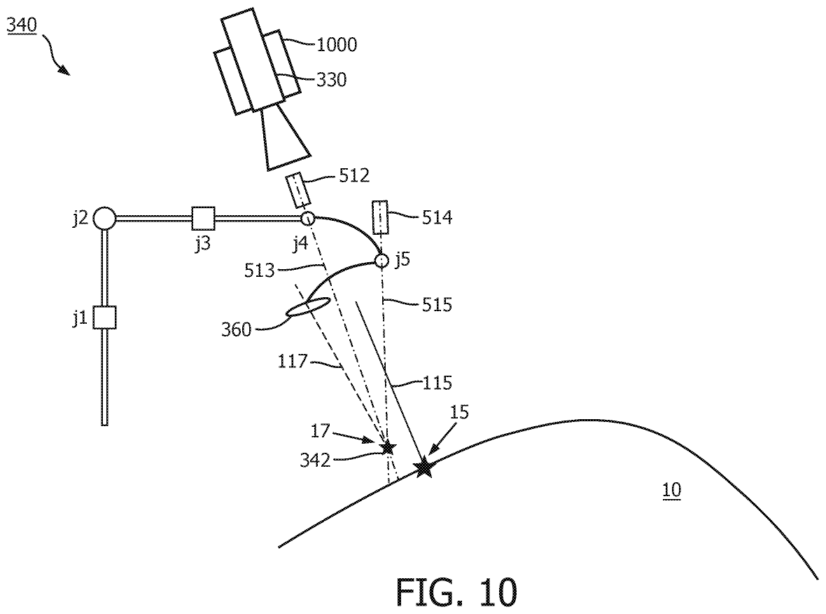

FIG. 10 illustrates a portion of a third version of robotic system 20 of FIG. 1. The third version of the robotic system 20 as illustrated in FIG. 10 is similar in construction and operation to the first version illustrated in FIG. 3 and described in detail above, so for the sake of brevity only differences therebetween will now be described.

In the third version of robotic system 20, camera 330 is actuated by a motor 1000 such that it can be maneuvered and positioned along planned path 115. Here again it is assumed that camera 330 is registered to preoperative images. In the case of the third version illustrated in FIG. 10, the projection of end-effector 42 onto captured images, reflecting the situation when end-effector 42 is aligned and oriented to planned entry point 15 along planned path 115, is a parallel projection. For example, if the shape of end-effector 42 is circular, then the projection is also circular. In that case, controller 50 can be configured to control the position of end-effector 42 so that a parallel projection is detected in the captured images, which is a unique solution. This can be done before or after RCM 342 is aligned to entry point 15. If it is done before, then RCM 342 can be positioned by aligning the center of the projection of end-effector 42 in the plan overlay and the detected position of end-effector 42 in the captured images.

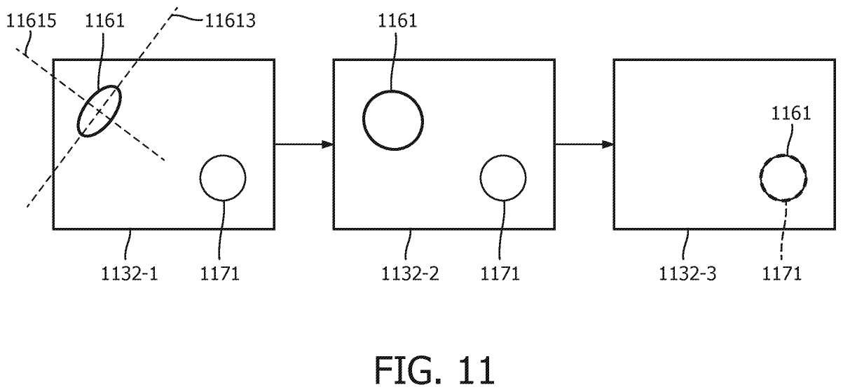

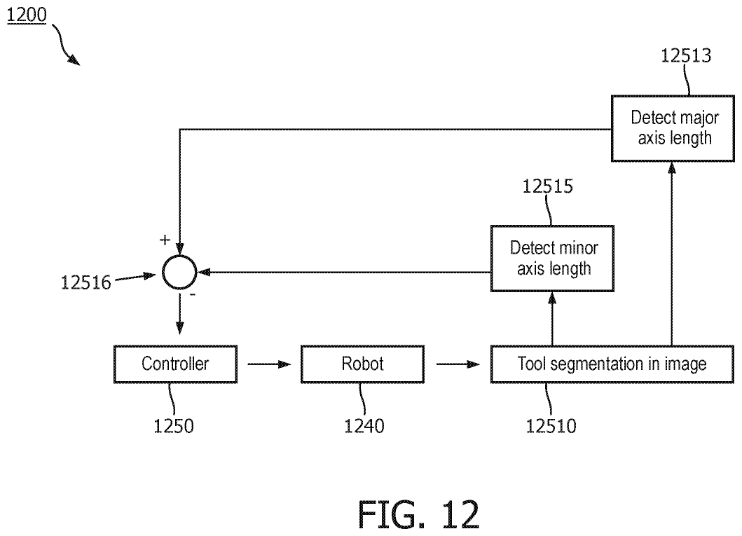

FIG. 11 illustrates a process of alignment and orientation of a circular robot end-effector 42 to a planned position for the robot end-effector 42 using a series of video frames captured by camera 330 using the third version of robotic system illustrated in FIG. 12.

Here in a first captured video frame 1132-1 captured by camera 330 is shown a projection 1171 of end-effector 42 as it should appear in video frame 1132-1 if end-effector 42 was aligned and oriented to planned entry point 15 along planned path 115. Instead, however, the detected image 1161 of end-effector 42 has an elliptical shape with a major axis 11613 and a minor axis 11615, and is laterally displaced from the position of projection 1171.

In a second frame 1132-2 captured by camera 330 is shown the detected image 1161 of end-effector 42 now has a circular shape as a result of a control algorithm executed by robot controller 50 to control the RCM mechanism of robot 40 to cause the detected image 1161 of end-effector to have a circular shape. However, it is seen in second frame 1132-2 that detected image 1161 is still laterally displaced from the position of projection 1171 and is larger in size than projection 1171.

After the situation depicted in video frame 1132-2 has been reached, the RCM mechanism (e.g., joints j4 and j5) of robot 340 can be locked and the positioning mechanism moved to align the RCM with the planned entry.

Since both shapes are now in parallel projection, in this step, only the centroids need to be aligned, for example using a method described in U.S. Pat. No. 8,934,003 B2. Once the centroids are aligned, the scale has to be aligned (the size of the circle of detected end-effector 42 to the size of the projected end-effector 42 according to the plan). The scale is defined by the motion of the robot 40 along tool path 115 which can be computed in the positioning mechanism coordinate frame.

In a third frame 1132-3 captured by camera 330 is shown the detected image 1161 of end-effector 42 is now aligned with projection 1171.

FIG. 12 illustrates one example embodiment of another feedback loop 1200 which may be employed in an operation of method of robot-based guidance. FIG. 12 illustrates one example embodiment of a feedback loop 1200 which may be employed in an operation or method of robot-based guidance which may be executed, for example, by robotic system 20. Various operators of feedback loop 1200 are illustrated as functional blocks in FIG. 812. Feedback loop 1200 involves a controller 1240, a robot 1250, a tool segmentation operation 12510, a major axis detection operation 12513, a minor axis detection operation 12515, and a processing operation 12516. Here, feedback loop 1200 is configured to operate with a reference object (e.g., end-effector 42) having an elliptical projection (e.g., a circular shape). In some cases, tool segmentation operation 12510, major axis detection operation 12512, minor angle detection operation 12515, and processing operation 12516 may be performed in hardware, software, firmware, or any combination thereof by a robot controller such as robot controller 50.

An example operation of feedback loop 1200 will now be described.

Processing operation 8516 subtracts the detected center and angle of a captured image of end-effector 42 from a target angle and a target center for end-effector 42, resulting in two error signals: a center error and an angle error. Processing operation 8516 combines those two errors (e.g. adds them with corresponding weights) and supplies the weighted combination as a feedback signal to controller 1250, which may be included as a component of robot controller 50 discussed above. Here controller 1250 may be a proportional-integral-derivative (PID) controller or any other appropriate controller known in art, including a non-linear controller such as a model predictive controller. The output of controller 1250 is a set of RCM mechanism joint velocities. The mapping to joint velocities can be done by mapping yaw and pitch of the end-effector 42 of robot 1240 to x and y coordinates in the captured images. The orientation of end-effector 42 can be mapped using a homography transformation between the detected shape of end-effector 42 in the captured images, and the parallel projection of the shape onto the captured images.

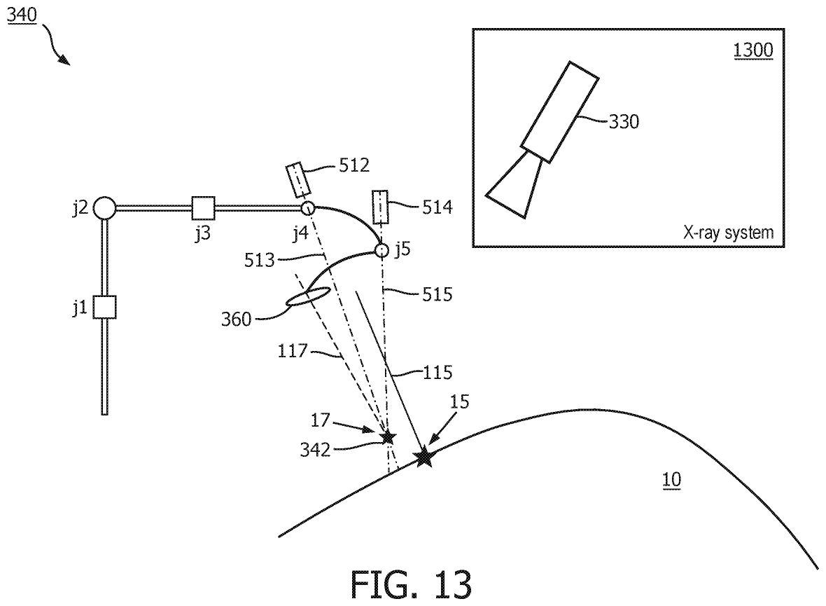

FIG. 13 illustrates a portion of a fourth version of robotic system 20 of FIG. 1. The third version of robotic system 20 as illustrated in FIG. 13 is similar in construction and operation to the first version illustrated in FIG. 3 and described in detail above, so for the sake of brevity only differences therebetween will now be described.

In the third version of robotic system 20, camera 330 is mounted on an intraoperative X-ray system 1300 which is configured to generate a rotational 3D scan where planned path 115 is located.

Other versions of robotic system 20 are possible. In particular, any of the versions described above with respect to FIGS. 3, 9, 10, etc. may be modified to include intraoperative X-ray system 1300.

While preferred embodiments are disclosed in detail herein, many variations are possible which remain within the concept and scope of the invention. Such variations would become clear to one of ordinary skill in the art after inspection of the specification, drawings and claims herein. The invention therefore is not to be restricted except within the scope of the appended claims.

1. A system, comprising:
a robot having a remote center of motion (RCM) mechanism with two motor axes, and an end-effector at a distal end of the robot;
a light projection apparatus configured to project two or more light beams intersecting at the RCM;
an imaging system configured to capture images of the RCM mechanism in a field of operation including a planned entry point and a planned path through the RCM; and
a robot controller configured to control the robot and position the RCM mechanism, the robot controller including an image processor which is configured: to receive the captured images from the imaging system, to register the captured images to three-dimensional (3D) pre-operative images, to define an entry point and a path for the RCM in the captured images using the projected light beams, and to detect and track in the captured images a reference object having a known shape,
wherein the robot controller is configured to: compute robot joint motion parameters, in response to the defined entry point, the defined path, and the detected reference object, which align the end-effector to the planned entry point and the planned path; to produce robot control commands, based on the computed robot joint motion parameters, which align the end-effector to the planned entry point and the planned path; and to communicate the robot control commands to the robot, and
wherein the robot controller is configured to compute the robot joint motion parameters by: determining one or more geometric parameters of the reference object in the captured images, and aligning the one or more geometric parameters of the reference object in the captured images to one or more corresponding known geometric parameters of the reference object as they appear to the imaging system when the reference object is located at a planned position of the reference object.

2. The system of claim 1, wherein the image processor is configured to detect the entry point as an intersection of the projected light beams, and wherein the robot controller is configured to control the robot to align the intersection of the projected light beams with the planned entry point.

3. The system of claim 1, wherein the image processor is configured to: project the known shape of the reference object at the planned position onto the captured images, and wherein the robot controller is configured to control the robot to overlay the detected reference object in the captured images with the projected known shape.

4. The system of claim 1, wherein the imaging system is configured to capture two-dimensional (2D) images of the RCM mechanism in the field of operation from a plurality of cameras spaced apart in a known configuration, and wherein the image processor is configured to detect and track the reference object having the known shape in the captured 2D images from each of the plurality of cameras, and to reconstruct a 3D shape for the reference object from the captured 2D images.

5. The system of claim 1, wherein the RCM mechanism is configured to rotate the end-effector about an insertion axis passing through the planned entry point, and wherein the end-effector has a feature that defines its orientation in a plane perpendicular to the insertion axis, wherein the image processor is configured to detect the feature in the captured images and to project a planned position of the feature onto the captured images, and wherein the robot controller is configured to control the robot to align the detected feature and the planned position.

6. The system of claim 1, wherein the reference object is the end-effector.

7. The system of claim 4, wherein the imaging system includes a camera and an actuator for moving the camera, wherein the camera is positioned by the actuator along the planned path, and wherein the robot controller is configured to control a position of the end-effector so that the image processor detects a parallel projection of the end-effector.

8. The system of claim 1, wherein the imaging system includes an X-ray system configured to generate a rotational three-dimensional (3D) scan of the planned path.

9. A method, comprising:
providing at least two light beams which intersect at a remote center of motion (RCM) defined by an RCM mechanism of a robot having an end-effector at a distal end thereof;
capturing images of the RCM mechanism in a field of operation including a planned entry point and a planned path through the RCM;
registering the captured images to three-dimensional (3D) pre-operative images;
defining an entry point and a path for the RCM in the captured images using the projected light beams;
detecting and tracking in the captured images a reference object associated with the end-effector, the reference object having a known shape;
in response to information about the entry point, the path, and the reference object, computing robot joint motion parameters which align the end-effector to the planned entry point and the planned path; and
communicating robot control commands to the robot, based on the computed robot joint motion parameters, which align the end-effector to the planned entry point and the planned path,
wherein computing the robot joint motion parameters includes determining one or more geometric parameters of the reference object in the captured images and aligning the one or more geometric parameters of the reference object in the captured images to one or more corresponding known geometric parameters of the reference object as they appear to the imaging system when the reference object is located at a planned position of the reference object.

10. The method of claim 9, including detecting the entry point as an intersection of the projected light beams, and controlling the robot to align the intersection of the projected light beams with the planned entry point.

11. The method of claim 9, including:
projecting the known shape of the reference object at the planned entry point onto the captured images;
and
controlling the robot to overlay the detected reference object in the captured images with the projected known shape.

12. The method of claim 9, including:
capturing two-dimensional (2D) images of the RCM mechanism in the field of operation from a plurality of cameras spaced apart in a known configuration; and
detecting and tracking the reference object having the known shape in the captured 2D images from each of the plurality of cameras; and
reconstructing a 3D shape for the reference object from the captured 2D images.

13. The method of claim 9, including:
rotating the end-effector about an insertion axis passing through the planned entry point, wherein the end-effector has a feature that defines its orientation in a plane perpendicular to the insertion axis;
detecting the feature in the captured images;
projecting a planned position of the feature onto the captured images; and
controlling the robot to align the detected feature and the planned position.

14. The method of claim 9, including:
capturing the images of the RCM mechanism using a camera positioned along the planned path, wherein the reference object is the end-effector; and
controlling a position of the end-effector so that a parallel position of the end-effector is detected in the captured images.

15. A robot controller for controlling a robot having a remote center of motion (RCM) mechanism with two motor axes and an end-effector at a distal end of the robot, the robot controller comprising:
an image processor which is configured: to receive captured images of the RCM mechanism in a field of operation including a planned entry point and a planned path through the RCM, to register the captured images to three-dimensional (3D) pre-operative images, to define an entry point and path for the RCM in the captured images, and to detect and track in the captured images a reference object associated with the end-effector, the reference object having a known shape; and
a robot control command interface configured to communicate robot control commands to the robot,
wherein the robot controller is configured to compute robot joint motion parameters, in response to the defined entry point, the defined path, and the detected reference object, which align the end-effector to the planned entry point and the planned path, and is further configured to produce the robot control commands, based on the computed robot joint motion parameters, which align the end-effector to the planned entry point and the planned path,
wherein the robot controller is configured to compute the robot joint motion parameters by: determining one or more geometric parameters of the reference object in the captured images, and aligning the one or more geometric parameters of the reference object in the captured images to one or more corresponding known geometric parameters of the reference object as they appear to the imaging system when the reference object is located at a planned position of the reference object.

16. The robot controller of claim 15, wherein the image processor is configured to detect the entry point as an intersection of the projected light beams, and wherein the robot controller is configured to control the robot to align the intersection of the projected light beams with the planned entry point.

17. The robot controller of claim 15, wherein the image processor is configured to project the known shape of the reference object at the planned position onto the captured images, and wherein the robot controller is configured to control the robot to overlay the detected reference object in the captured images with the projected known shape.

18. The robot controller of claim 15, wherein the image processor is configured to receive two-dimensional (2D) images of the RCM mechanism in the field of operation from a plurality of cameras spaced apart in a known configuration, to detect and track the reference object having the known shape in the captured 2D images from each of the plurality of cameras, and to reconstruct a 3D shape for the reference object from the captured 2D images

19. The robot controller of claim 15, wherein the RCM mechanism is configured to rotate the end-effector about an insertion axis passing through the planned entry point, and wherein the end-effector has a feature that defines its orientation in a plane perpendicular to the insertion axis, wherein the image processor is configured to detect the feature in the captured images and to project a planned position of the feature onto the captured images, and wherein the robot controller is configured to control the robot to align the detected feature and the planned position.

20. The robot controller of claim 15, wherein the robot controller is configured to receive the captured images from a camera positioned by an actuator along the planned path, and wherein the robot controller is configured to control a position of the end-effector so that the image processor detects a parallel projection of the end-effector.
